(19) United States
(12) Patent Application Publication
(10) Pub. No.: US 2007/0056587 A1
(43) Pub. Date: Mar. 15, 2007

(54) MEDICAL DEVICE FOR A PATIENT'S VENTILATORY SUPPORT

(75) Inventor: Maurizio Travan, Mirandola (Modena) (IT)

Correspondence Address: AKERMAN SENTERFITT, P.O. BOX 3188, WEST PALM BEACH, FL, 33402-3188, US

(73) Assignee: DEAS S.r.l., Castel Bolognese (Ravenna) (IT)

(21) Appl. No.: 11/468,060

(22) Filed: Aug. 29, 2006

(30) Foreign Application Priority Data
Sep. 1, 2005 (EP) 05425615.1

(51) Int. Cl. A61M 16/00

(52) U.S. Cl. 128/204.18; 128/204.28

(57) ABSTRACT

A ventilatory support device with administration of gas have a predetermined content of oxygen in continuously positive pressure. The device comprises a pipe extending between an intake fitting and a delivery fitting of the gas, wherein an inner portion of the pipe diverges towards the delivery fitting. The device also comprises a substantially tubular injector supported in the pipe and orientated towards the delivery opening, and suitable for being connected to a pressurised gas source having a predetermined oxygen content.

### FIELD OF THE INVENTION

The present invention refers to a medical device for a patient's ventilatory support.

More in particular, the present invention refers to a ventilatory support device with administration of gas having a predetermined content of oxygen in continuously positive pressure, also called CPAP (continuous positive air pressure) assisted respiration device.

### BACKGROUND OF THE INVENTION

The continuous positive air pressure (CPAP) ventilatory support process represents a now well established method for the treatment of respiratory illnesses.

As is known, CPAP assisted respiration requires the patient's efficient spontaneous respiration and is used to treat illnesses of the hyaline membranes, wherein the alveoli have greater difficulty in expanding during inspiration and collapse more easily during expiration.

In particular, the loss of alveolar tone associated with other causes such as intraalveolar transudation, interstitial oedema and the reduction or lack of surface-active substance lead to a series of modifications of the respiratory mechanics and of the distribution of air in the lungs that result in serious and progressive respiratory insufficiency.

For this reason, to at least partially support the alveolar distension, a gas mixture, with a suitable oxygen content dosed in litres per minute (L/min) is administered to the patient that allows a pressure gradient to be obtained between the inside of the respiratory tree and the outside, active for the entire duration of the respiratory cycle.

The pressure gradient has the purpose of allowing a greater expansion of the alveoli during inspiration, preventing alveolar collapse at the end of expiration, keeping a determined positive end expiration pressure (PEEP), as well as reducing the respiration effort thus avoiding the occurrence of complications such as hypoxaemia, hypercapnia, metabolic and respiratory acidosis.

A known device commonly used for CPAP respiration comprises a main pipe extending along a given axis to connect an oxygen intake opening and a delivery opening suitable for being connected to a patient.

The intake opening is suitable for being connected to a pressurised gas source having a predetermined oxygen content.

Generally, to place the delivery opening in communication with the patient, a face mask or else a helmet is provided that are connected to the body of the device.

To ensure that a sufficient continuously positive pressure is maintained in the main pipe, the device is equipped with a valve, called PEEP valve, which allows a predetermined positive pressure to be generated in the entire system at the end of expiration.

Known mechanical or else hydraulic valves are used as PEEP valves.

In the prior art, normally to ensure ventilatory support it is necessary to ventilate the patient with a flow of gas of between about 60 and 120 litres per minute (L/min). The flow of gas at the delivery opening inspired on average by the patient is overall equal to 6-12 litres per minute.

The overall flow of gas pumped into the main duct is between about 60 and 120 litres per minute for the following reason.

During inspiration patient's instantaneous flows comprised between about 100 and 110 litres per minute occur.

The pumping of a high overall flow of gas does indeed ensure ventilatory support also when such instantaneous flows occur, and thus avoids the occurrence of undesired depressions in the system.

The known ventilatory support device, whilst allowing the desired positive pressure to be obtained during the entire respiration cycle, does nevertheless have some drawbacks that have not been overcome yet.

The main drawback lies in the that it is necessary to use hydraulic and mechanical valves that have some drawbacks linked to the wear and to the inertia of the mechanical parts and to the pollution by the water in the case of hydraulic valves, and they are also made up of a large number of components.

Another drawback is represented by the high consumption of gas (60-120 litres per minute compared to 6-12 litres per minute inspired on average by the patient).

As stated above, with the known device, it is indeed necessary to overdose the flow of gas to adapt the system to high instantaneous flows during inspiration.

Such drawback is particularly clear under emergency conditions or during transportation of the patient, i.e. when the gas is supplied only by a low-capacity cylinder.

### SUMMARY OF THE INVENTION

It is an object of the present invention to make a ventilatory support device of the aforementioned type that allows the aforementioned drawbacks to be overcome.

In particular, it is an object of the present invention to provide a device that is simple to be manufactured and able to ensure a positive end expiration pressure without requiring the help of mechanical or hydraulic valves and with a reduced overall gas consumption.

It is a further object of the present invention to control the flow of gas and to adapt the system to high instantaneous flows during inspiration.

In particular, it a object of the invention to make a device which is able to recover a positive overall gas flow pressure in the main pipe and of exploiting the pressure of the recovered gas as end expiration valve.

According to one aspect of the present invention, a ventilatory support device with administration of gas having a predetermined content of oxygen in continuously positive pressure comprises a pipe extending between a gas intake fitting and a gas delivery fitting, wherein an inner portion of the pipe diverges towards the gas delivery fitting, and a substantially tubular injector supported in said pipe and orientated towards the delivery opening, said injector being suitable for being connected to a gas source having a predetermined oxygen content.

According to one embodiment of the invention, the device comprises a discharge opening for discharging expired gas, the discharge opening being open in the main pipe and positioned in a zone between the injector and the delivery fitting at a predetermined distance away from the injector.

According to a further aspect of the invention, the injector is arranged substantially coaxial to the axis of the main pipe and substantially opposite the mouth of the delivery fitting in such a manner to face the mouth of the delivery fitting.

Further preferred embodiments of the invention are defined in the dependent claims.

Further characteristics and advantages of the invention shall become more evident from the following description of an embodiment thereof, given by way of indicative and non limiting example with reference to the attached figures.

### BRIEF DESCRIPTION OF THE DRAWINGS

FIG. 1 illustrates a perspective view of a device according to one embodiment of the invention connected to two sources of oxygen and pressurised air.

FIG. 2 illustrates an enlarged view of the device of FIG. 1.

FIG. 3 illustrates a further enlarged view of the device of FIG. 1.

FIG. 4a illustrates view from above of a front portion of the device illustrated in FIG. 3.

FIG. 4b illustrates a section of the portion of FIG. 4a.

FIG. 5 illustrates a section of the device of FIG. 3.

FIGS. 6a-6c illustrate sections of the device of FIG. 1 wherein the gas flows during a respiration cycle are indicated.

FIG. 7a illustrates an perspective view of a rear portion of the device illustrated in FIG. 3.

FIG. 7b illustrates a section of the rear portion illustrated in FIG. 7a.

FIG. 8 illustrates a filter for the device of FIG. 1.

FIG. 9 illustrates the same section of FIG. 5 wherein some relevant dimensions are indicated.

### DETAILED DESCRIPTION

With reference to the attached figures, a ventilatory support device in continuously positive air pressure (CPAP) is indicated with reference number 10.

Preferably, the entire device 10 is of the disposable type and it is made entirely of plastic.

As it shall be better illustrated hereafter, in the illustrated solution, the device 10 is associated with a system 13 for feeding pressurised oxygen comprising a pure oxygen source 11 and an air source 12 having a known oxygen content.

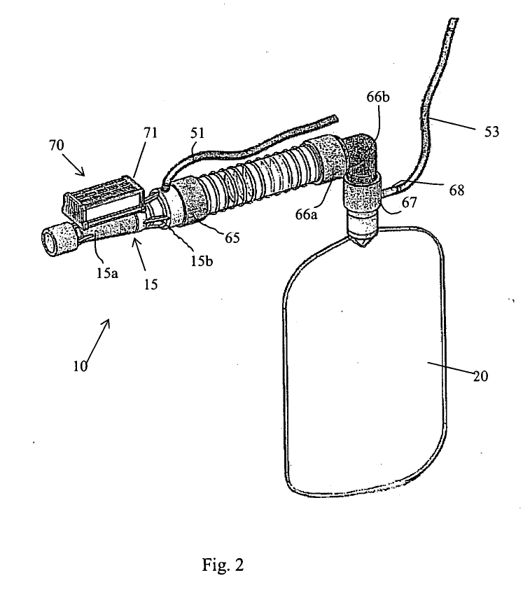

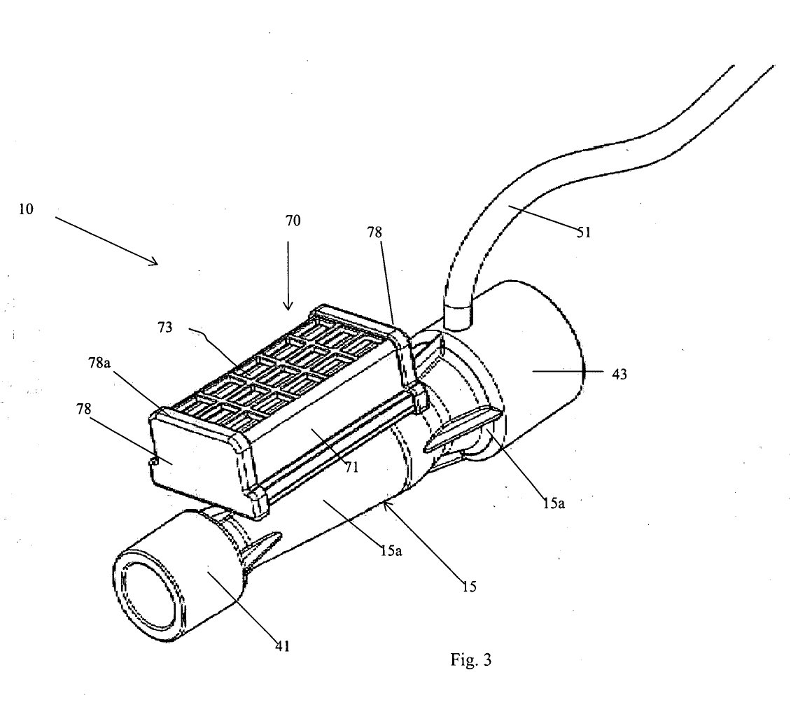

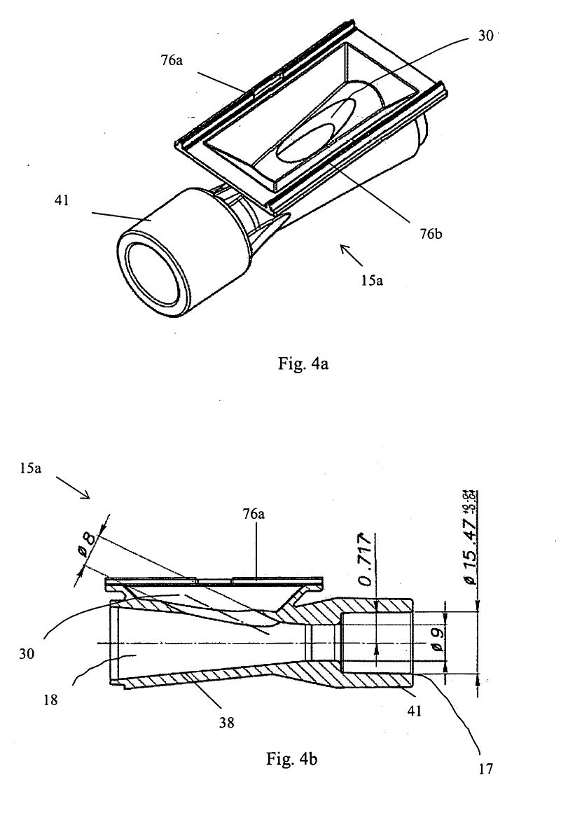

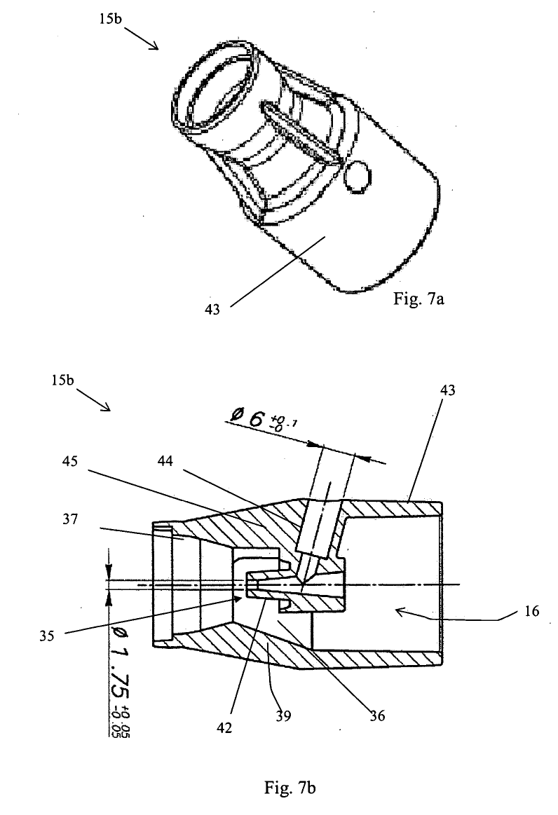

Hereafter in the description, by way of example, reference will be made to the supply system, even if nothing forbids that the device 10 can be associated with a single pure oxygen source or with a single air source. Even more specifically, with reference to FIGS. 2-5 and 9, the device 10 comprises a substantially tubular shaped body 15 that is made, in the case of the illustrated solution, of a front portion 15a (FIGS. 4a, 4b) and a rear portion 15b (FIGS. 3, 7a, 7b) joined together.

In the body 15, the device 10 comprises a substantially rectilinear pipe 18 that extends along a determined axis X between an intake fitting, indicated with reference number 16 and formed in the rear portion 15b, and a delivery fitting 17 formed in the front portion 15a and that is suitable for being connected to a patient.

The patient can be connected directly to the delivery fitting 17, or else through a face mask not illustrated in the drawings that is fixed to the delivery fitting 17.

The delivery fitting 17 is substantially aligned along the axis X with the intake fitting 16.

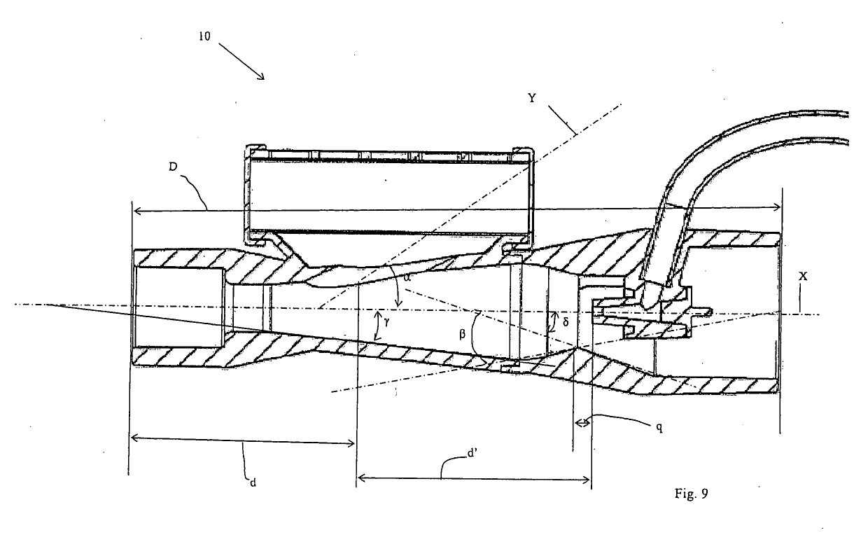

Preferably, the main pipe 18 is 123.5 mm long (length indicated with D in FIG. 9) and the intake fitting 16 and the delivery fitting 17 have a circular section with a diameter of 25 mm and about 15.5 mm respectively.

The device 10 comprises a substantially tubular injector 35 supported in the pipe 18 and orientated towards the delivery fitting 17. The injector 35 is substantially coaxial to the axis X of the main pipe 18 and it defines a substantially annular port 36 in the intake fitting 16 around the injector 35.

In practice, the device 10 comprises an injector 35, which is arranged along the axis X in the intake fitting 16 and defines an annular port 36 that coaxially surrounds the injector 35 itself.

Downstream of the injector 35, the main pipe 18 comprises a diverging inner portion 37, which diverges towards the delivery fitting 17.

The injector 35 is therefore upstream of the diverging portion 37 and substantially opposite to the mouth of the delivery fitting 17, at a relatively short distance away from it, in such a way to face said mouth.

Preferably, the distance is equal to about four times the diameter of the mouth of the delivery fitting 17, i.e. about 80 mm in the case of the illustrated solution.

The tubular injector 35 is connected to the pressurised oxygen sources 11 and 12, whereas the intake fitting 16 is placed in communication with an oxygen or oxygen and air reserve 20.

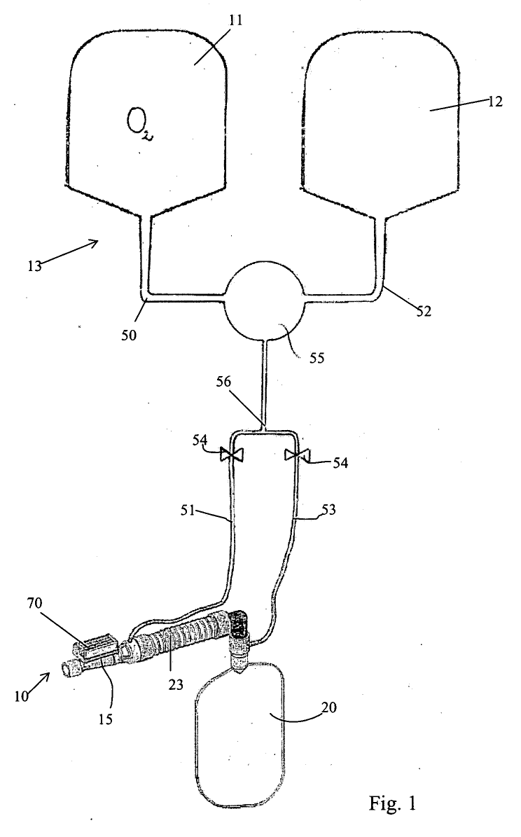

In the illustrated solution the gas reserve 20 is in the form of an elastic sack (FIGS. 1 and 2) containing an air/oxygen mixture of suitable content that is connected, as it shall be explained more clearly hereafter, to the intake fitting 16 through a spiral-shaped tube 23.

The device 10 also comprises a discharge opening 30, which is open in the main pipe 18 in a zone between the injector 35 and the mouth of the delivery fitting 17 and which, as it shall be explained more clearly hereafter, discharges the gas expired by the patient in the expiration step.

In the case of the illustrated solution, the discharge opening 30 is placed at a predetermined distance d′ away from the mouth of the injector 35 indicated in FIG. 9, substantially equal to the distance d from the mouth of the delivery fitting 17, wherein d is between 42 and 46 mm, and even more preferably it is equal to 43.95 mm.

Preferably, the discharge opening has an axis Y forming an angle α with the axis of the main pipe X.

The two axes X and Y converge towards the delivery fitting 17. The angle α is between about 22 and about 30 degrees, and even more preferably 26 degrees.

It should also be observed that the main pipe 18, starting from the intake fitting 16, has a Venturi portion, i.e. it comprises a narrowing 40 made of a first converging portion 39, which surrounds the injector 35 in the rear fitting 15b of the body 15, and the aforementioned diverging portion 37.

In the illustrated solution, the main pipe 18 also comprises a third converging portion 38 that extends for at least ½, preferably for about ⅔, of the length of the main pipe 18 between the tubular injector 35 and the delivery fitting 17.

The discharge opening 30 is made in a substantially middle area of the third converging section 38, comprised between the tubular injector 35 and the mouth of the delivery fitting 17.

The diverging portion 37, which is made in the rear fitting 15b of the body 15, is connected directly to the third converging portion 38.

Preferably, the third converging portion 38 is longer than the first converging portion 39 and the diverging portion 37.

Preferably, the ratio between the length of the third converging portion 38, of the first converging portion 39 and of the diverging portion 37 is equal to about 4:1, 5:1.

In the case of the illustrated solution, the third converging portion 38 is about 48.3 mm long and forms a converging angle γ with the axis X of the main pipe 18, which is between about 4.500 and 7.000 degrees, and is preferably equal to 5.611 degrees.

The first converging portion 39 is about 14.5 mm long and forms a converging angle δ with the axis X of the main pipe 18 of between about 18 and 22 degrees, preferably 20 degrees.

It should be noted that the first converging portion 39 thus made allows the area of the mouth of the intake fitting 16 to be taken to such a value that the ratio between area of the outlet section of the injector 35 and the area of the minimum circular section of the converging portion 39 is between 1:50 and 1:150.

The diverging portion 37 is about 11.1 mm long and forms a diverging angle β with the axis X of the main pipe 18 of between about 13 and 17 degrees, preferably 15 degrees.

It should be also noted, as stated above, that only the first converging portion 39 of the Venturi portion surrounds the tubular injector 35. Basically, the tubular injector 35 is in withdrawn position with respect to the diverging portion 37 towards the mouth of the intake fitting 16.

Preferably, the distance q between the mouth of the injector 35 and the third diverging portion 37 is between 3 and 4 mm and this, as it shall be explained more clearly hereafter, allows a larger space to be obtained around the tubular injector 35 for the drawing of air.

It should also be noted that, in the illustrated solution, the pipe 18 comprises, near the mouth of the delivery fitting 17, an end portion 41 with constant section and, similarly, an end portion 43 with constant section near the intake fitting 16. These portions with constant section allow the device 10 to be respectively fitted to the spiral tube 23 and to a face machine, not illustrated in the drawings.

With reference to FIG. 7a, the injector 35 shall now be illustrated in detail.

The injector 35 comprises a frustum of cone portion 42 that is integral in a single body with the end portion 43 of the main pipe 18. The connection with the end portion 43 is made through a connection portion 45, which partially occupies the annular port 36.

In the portion 15b of the body 15 a passage 44 is also formed that is placed in fluid communication with the frustum of cone portion 42 on a side thereof to allow the intake of pressurised oxygen. The intake takes place through a flexible cannula 51 that is fixed to the passage 44.

As far as the oxygen feed is concerned, as stated above, the device 10 is connected to the oxygen/pressurised air feeding system 13 (FIG. 1), which is of the fixed type and usually arranged on a wall of a room and comprises the aforementioned sources 11 and 12.

The system 13 comprises a first line 50 and a second line 52 that connect the sources 11 and 12 to the device 10. Preferably, the device 10 is provided with a mixer 55, arranged downstream of the lines 50 and 52, intended to mix oxygen and air coming from the two sources 11 and 12, so as to obtain an air flow having a determined oxygen content.

The device 10 is also provided with a T-like joint, schematically indicated in FIG. 1 with reference number 56, which is arranged downstream of the mixer 55 and is suitable for dividing the flow of mixed gases respectively towards the injector 35 and the sack 20.

The T-like joint is connected to the injector 35 through the aforementioned cannula 51 and to the sack 20 through a cannula 53. The latter is connected to the sack 20 through a small tube 68.

The two cannulas 51 and 53 are fitted with flow regulators 54 for the regulation of the pressure/flow rate of the gas flow.

The connection of the sack 20 to the body 15 is completed by the aforementioned spiral tube 23 and by fittings 65, 66a, 66b and 67 (FIG. 2) of the known type.

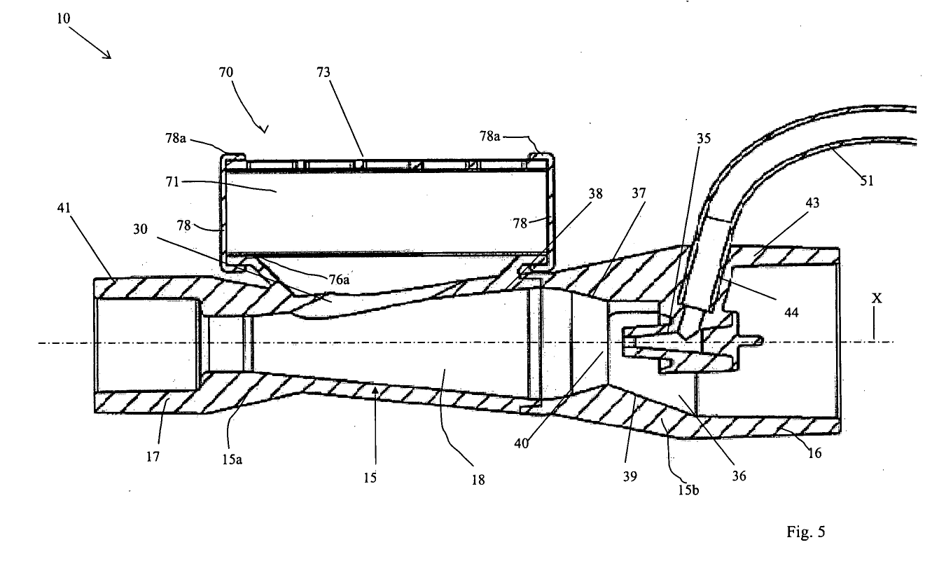

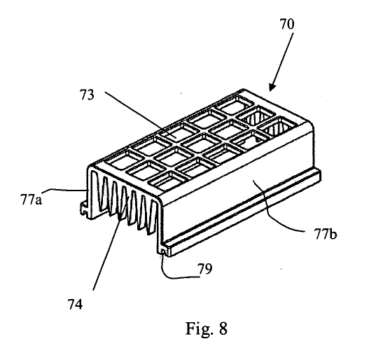

According to another characteristic of the present invention, the device 10 comprises a filter 70 (FIGS. 5 and 8) fitted over the discharge opening 30 that is able to absorb the noise of the pressurised gas flows passing in the main pipe 18.

Preferably, the filter 70 comprises a box-shaped structure 71 (FIG. 8) fixed to the discharge opening 30 and provided with a grid 73. A pleated filtering body 74, preferably made from hydrophobic material capable of retaining the moisture contained in the gas expired by the patient, is fixed under the grid 73.

The pleated filter 74 also has an antibacterial function, i.e. it is able to protect the external environment from the possible emission of bacteria or microbes due to the patient's pathologies.

Preferably, the device 10 is provided with mutual engagement means to removably fix the filter 70 onto the discharge opening 30.

For such purpose the device 10 comprises opposite ribs 76a, 76b made integrally with the discharge opening 30 and that engage in corresponding grooves 79 formed on opposite sides 77a, 77b of the structure 71.

The filter 70 is completed by side covers 78 that laterally close the remaining sides of the structure 71 and engage through respective edges 78a at the ribs 76a, 76b and on the grid 73.

At any rate, it is understood that the filter 70 can be replaced by another filtering member having different properties about noise absorption and protection from bacteria.

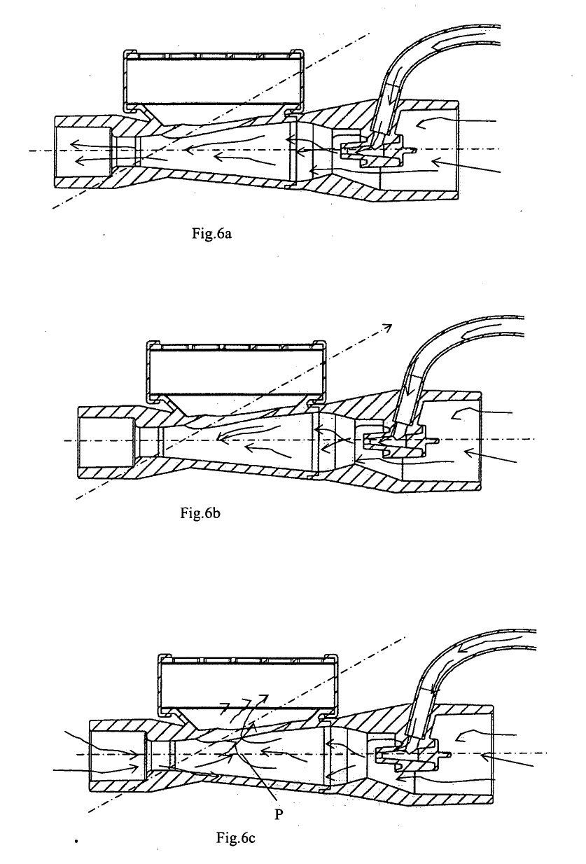

With reference to FIGS. 1 and 6a-6c, the operation of the device 10 shall now be described.

Initially, once the device 10 has been connected to the air and oxygen sources 11 and 12, a step of setting the pressure of the gases is carried out by acting on the valves 54, so as to obtain a predetermined flow rate of the air with a known oxygen content to the injector 35 and to the sack 20.

The operating pressure range is preferably between 0.1 and 1 bar. The air is divided between the sack 20 and the injector 35 so that the flow rate of the air introduced into the sack 20 is equal to about 2-3 times that of the injector 35.

The flow rate of the jet coming out from the injector 35 varies between a minimum of 15 litres/minute and a maximum of 30 litres/minute, according to the patient's needs.

The jet coming out from the injector 35 is able to draw the air/oxygen gas mixture into the main pipe 18 from the sack 20 through the intake fitting, thus allowing a satisfactory amplification of the total gas flow to the delivery outlet 17 with respect to the flow coming out from the injector 35.

The overall flow is relatively low with respect to gas flows in the devices of the prior art and it is between about 35 and 60 litres/minute.

Downstream of the injector 35 a submerged jet consisting of a flow coming out from the injector 35 and whose current lines are not defined by any solid wall (at least in a region near the outlet) is obtained.

The main characteristic of this jet is the drawing action exerted thereby with respect to the surrounding fluid.

The amplification of the flow is also completed by the suction action of the gas through Venturi effect.

Basically, the amplification of the flow is given by the sum of two actions: a first action is the forcing and drawing of the gas in front of and surrounding the injector nozzle (submerged jet) and a second action is the suction of the gas situated behind the injector nozzle through Venturi effect.

FIG. 6a illustrates the initial situation wherein the jet of air with known oxygen content is submerged in the gas flow drawn from the sack 20 through the intake fitting.

The drawing effect by the flow coming out from the injector 35 initially reduces the overall pressure of the flow in the diverging portion 37 of the main pipe 18, up to a negative value.

According to the one aspect of the invention, the flow downstream of the injector, i.e. in the zone of the main pipe 18 between the injector 35 and the delivery fitting, recovers the pressure that has been lost in the drawing step, thus allowing a satisfactory pressure value to be obtained in the main pipe 18.

An increase in the overall pressure of the flow of between about 2 and 16 cm of H₂O column is thus obtained in the main pipe 18.

It is to be noted that the positive pressure is completely recovered in a middle zone of the third converging portion 38 at the discharge opening 30.

It goes without saying that the greater the pressure value of the jet coming out from the injector 35 is, the greater the overall recovered pressure value is.

The device 10 is connected to a patient's respiratory apparatus for example to the mouth with the help of a face mask.

During inspiration, the patient creates a depression that, through the delivery fitting 17, propagates in the pipe 18, altering the ratio of the pressures generated therein by the jet.

This alteration involves an almost instant increase in the flow rate gain of the device that “following” the inspiration depression tries to keep the pressure positive, aim of the therapy.

It should be noted that, thanks to the recovery of pressure, the “following” of the inspiration depression and therefore the increase in flow occurs almost instantaneously.

In other words, when, during inspiration, there are sudden changes of pressure equal to instantaneous flows of about 80-110 L per minute that alter the overall pressures at stake in the system, there is an almost instantaneous increase in the gas flow/pressure induced in the main pipe 18 through the opening 16 that satisfies the variations in pressure/instantaneous flows needed in the delivery fitting 17.

Basically, there is a modulation of the flow rate of the gas induced in the main pipe based upon the patient's instantaneous inspiration flows. The system thus dynamically adapts to the patient's inspiration.

This substantially instantaneous “following” of the inspiration depression is also promoted by the aforementioned arrangement of the injector 35 relatively close, and opposite, to the delivery fitting 17.

It should be noted that the aforementioned reduction of the overall gas flow in the main pipe of between about 35 and 60 litres per minute is strictly correlated to the dynamic adaptation of the system to the instantaneous flow variation generated by the patient.

In fact, the overall gas flow in the main pipe momentarily increases only when required (increase in the induced flow rate) in response to a variation generated by the patient, otherwise the flow is kept at the same values of between 35 and 60 litres/minute which are sufficient to ensure the patient's ventilatory support. The latter, as stated above, respires about 6-12 litres of gas per minute.

FIG. 6b illustrates the instant of switching from the inspiration step to the expiration step.

In the expiration step (FIG. 6c), the gas expired by the patient is placed in the main pipe 18 through the delivery fitting 17 in the opposite direction to the gas flow inlet. The expired gas counteracts the gas flow generated by the jet until a pressure equilibrium point is reached indicated with P in FIG. 6b.

The pressure equilibrium P is an unstable state of dynamic equilibrium and the two flows, in order to maintain such equilibrium, must come out through the discharge opening 30.

The deviation of the flows is also called flow switch.

Basically, the discharge opening 30 branches from the main pipe exactly where, in the expiration step, the overall recovery of pressure of the gas occurs and where the pressure equilibrium of the expired gas and of the inlet gas flow is reached, so that the two flows can deviate towards the discharge opening 30.

FIG. 6c illustrates such condition wherein the two flows deviate to the outside through the filter 70.

At the end of the respiratory cycle, or rather during the pause that occurs between one respiratory cycle and the next, the pressure remains at a positive value (PEEP) corresponding to that reached in the state of dynamic equilibrium of the two flows (expiratory and generated by the jet).

It should be noted that thanks to the configuration of the illustrated device it is possible to regulate the inlet gas flow rate through the valves 54 within the aforementioned values, without altering the achievement of the pressure equilibrium at the discharge opening 30, and thus the positive end expiration pressure value, corresponding to 6-12 cm of H₂O column mentioned above.

The main advantage of the embodiment of the present invention is that of using the pressure recovery in the main pipe 18 to establish a state of dynamic equilibrium of the two flows in the main pipe, which constitutes an end expiration valve of the device.

The use of mechanical or hydraulic valves is thus avoided, thus obtaining a device having a simple structure with a small number of components.

A further advantage lies in the possibility of regulating, according to the patient's needs, the pressure of the jet coming out from the injector 35 and therefore the overall flow rate of the flow of gas in the pipe within a wide range of values.

In fact, by varying the pressure between a value of 0.2 and 1 bar, a satisfactory recovery of pressure is in any case ensured in the converging portion and therefore, in the expiration step, the desired pressure equilibrium that allows the deviation of the flows through the discharge opening is ensured.

In other words, it is possible to amplify the oxygen flow according to needs, without altering the pressures at stake in the device so as to ensure the establishment of the pressure equilibrium of the flows and their deviation into the discharge opening.

It should be observed that the desired pressure recovery is promoted by the substantially intermediate position of the discharge opening between the injector and the mouth of the delivery fitting.

A further advantage, with respect to devices of the prior art, is the low gas consumption (⅓-¼ of total consumption).

In fact, as mentioned above, the device allows a dynamic adaptation of the gas flow induced in the main pipe in response to pressure variations in the inspiration step, thus allowing less gas to be overall fed into the main pipe and without requiring an overdose.

In other words, the device is able to maintain a pressure/flow rate equilibrium inside the system that adapts to the variations in pressure and flow rate that occur during a respiratory cycle both in the inspiration step (increase in gas flow in response to instantaneous high inspiration flows) and in the expiration step (pressure equilibrium at the discharge opening).

A further advantage lies in the converging/diverging/converging configuration of the portions of the main pipe.

In fact, the diverging portion 37 promotes the entry of air, or gas mixture with known oxygen content, through the intake fitting and thus the amplification of the flow.

It should also be observed that the desired “gain” of the flow rate amplifier is promoted by the aforementioned sections ratio between the outlet mouth of the injector 35 and the smaller section of the first converging portion 39.

It should also be observed that the withdrawn position of the injector 35 with respect to the diverging portion 37 selected, as stated above, to have more space around the injector 35, further promotes the drawing of air.

Another advantage lies in the arrangement of the discharge opening having an axis that forms an angle converging with the axis of the main pipe. Such converging arrangement promotes the outlet of the flows in the expiration step.

A further advantage lies in the fact that the injector and the intake fitting can be connected to the same gas source. This allows an amplification of the flow to be obtained without varying the gas composition.

A further advantage lies in the presence of the filter mounted on the discharge opening that is able to absorb the noise of the pressurised gas flows as well as to retain the moisture contained in the gas expired by the patient.

A further advantage lies in the possibility, when both the pressurised sources are not available, but only the oxygen source is (e.g. under all emergency conditions or during transportation when only the oxygen cylinder is normally present), to feed the jet with the available source and ensure the necessary high flows, thanks to the amplification in flow rate carried out by the device.

Of course, a man skilled in the art can bring numerous modifications and variants to the ventilatory support device described above, in order to satisfy contingent and specific requirements, all of which are covered by the scope of protection of the invention, as defined by the following claims.

1. A ventilatory support device with administration of gas having a predetermined content of oxygen in continuously positive pressure, comprising:
a pipe extending between a gas intake fitting and a gas delivery fitting, wherein an inner portion of the pipe diverges towards the delivery fitting; and
a substantially tubular injector supported in the pipe and orientated towards the delivery opening, which is suitable for being connected to a pressurised gas source having a predetermined oxygen content.

2. The device of claim 1, further comprising a discharge opening for discharging expired gas, the opening being open in the main pipe and positioned in a zone between the injector and the delivery fitting at a predetermined distance away from the injector.

3. The device of claim 2, wherein the discharge opening is formed in a substantially middle zone between the tubular injector and the delivery fitting.

4. The device of claim 1, further comprising regulation means of the flow rate of the gas coming out from the injector to obtain a positive pressure of the gas with a known oxygen content downstream of the injector.

5. The device of claim 1, wherein the injector is arranged substantially coaxial to the axis of the main pipe and substantially opposite a mouth of the delivery fitting in such a manner to face the mouth of the delivery fitting.

6. The device of claim 5, wherein the injector is arranged at a distance equal to about four times the diameter of the mouth of the delivery fitting.

7. The device of claim 1, wherein, starting from the intake fitting towards the delivery fitting, the main pipe comprises a Venturi portion arranged around the tubular injector and made of a first converging portion and of said diverging inner portion.

8. The device of claim 7, wherein the tubular injector is arranged in a withdrawn position with respect to the diverging portion towards the intake fitting.

9. The device of claim 8, wherein the main pipe comprises a third portion that extends converging for at least half of the length of the main pipe that is situated between the tubular injector and the delivery fitting.

10. The device of claim 9, wherein the ratio between the length of the third converging portion, of the first converging portion and of the diverging portion is about 4:1.5:1, respectively.

11. The device of claim 9, wherein the third converging portion forms a converging angle with the axis of the main pipe, of between about 4.500 and 7.000 degrees.

12. The device of claim 9, wherein the third converging portion forms a converging angle with the axis of the main pipe, of about 6 degrees.

13. The device of claim 9, wherein the first converging portion forms a converging angle with the axis of the main pipe, of between about 18 and 22 degrees.

14. The device of claim 9, wherein the diverging portion forms a diverging angle with the axis of the main pipe of between about 13 and 17 degrees.

15. The device of claim 2, wherein the discharge opening has an axis forming an angle with the axis of the main pipe, wherein said axes converge towards the delivery opening.

16. The device of claim 15, wherein the angle between the axis of the discharge opening and the axis of the main pipe is between about 22 and about 30 degrees.

17. The device of claim 1, further comprising a reserve sack of gas with predetermined oxygen content that is connected to the intake opening.

18. The device of claim 1, further comprising means for connecting the tubular injector and the gas reserve to the same pressurised gas source.

19. The device of claim 18, further comprising means for fractionating the pressurised gas flow distributing it towards the injector and the gas reserve so as to maintain a concentration with known oxygen content.

20. The device of claim 2, further comprising a filter fitted over the discharge opening that is able to absorb the noise of the pressurised gas flows passing in the main pipe.

21. The device of claim 20, further comprising a membrane made of hydrophobic material capable of retaining the moisture contained in the gas expired by the patient.

22. The device of claim 21, further comprising mutual engagement means for removably fixing the filter to the discharge opening.

23. The device of claim 21, wherein said mutual engagement means comprises opposite ribs made integrally with the discharge opening and which engages in corresponding grooves formed on opposite sides of the filter.
